(19) United States
(12) Patent Application Publication
(10) Pub. No.: US 2008/0206894 A1
(43) Pub. Date: Aug. 28, 2008

(54) Bioresponsive Hydrogels

(75) Inventors: Louis Andrew Lyon, Marietta, GA (US); Jongseong Kim, Boston, MA (US)

Correspondence Address: TROUTMAN SANDERS LLP, 600 PEACHTREE STREET , NE, ATLANTA, GA, 30308, US

(73) Assignee: Georgia Tech Research Corporation, Atlanta, GA (US)

(21) Appl. No.: 11/915,501

(22) PCT Filed: May 24, 2006
(86) PCT No.: PCT/US06/20173

§ 371 Date: Nov. 26, 2007

Related U.S. Application Data
(60) Provisional application No. 60/683,917, filed on May 24, 2005.
(60) Provisional application No. 60/725,208, filed on Oct. 11, 2005.

(51) Int. Cl. G01N 33/544; C12Q 1/00

(52) U.S. Cl. 436/531; 436/528; 435/4

(57) ABSTRACT

The invention is a structure capable of detecting a target molecule, the invention including a functionalized polymer matrix having chemically reactive groups and at least one biomolecule attached to the chemically reactive groups to form a surface-modified bioresponsive polymer matrix.

### RELATED APPLICATIONS

This application is a 35 U.S.C. §371 U.S. National Stage Application of International Application Number PCT/US2006/020173 filed 24 May 2006, which claims, under 35 U.S.C. §119(e), the benefit of U.S. Provisional Application Ser. No. 60/725,208, filed 11 Oct. 2005, and U.S. Provisional Application Ser. No. 60/683,917, filed 24 May 2005, the entire contents and substance of which are hereby incorporated by reference as if fully set forth below.

### TECHNICAL FIELD

The invention is directed to a bioresponsive microstructure for use in biomolecular sensing.

### BACKGROUND OF THE INVENTION

Proteins and nucleic acids are information-rich molecules with structural and electrical properties that make them useful for manufacturing bioresponsive microstructures. Although microstructures are generally thought of as synthetic structures (e.g., hydrogels), they can be modified with biomolecules to produce a bioresponsive microstructure. One example of such a microstructure is a hydrogel. Over the past decade, a number of applications involving stimuli-sensitive hydrogels have arisen due to the great potential for hydrogels as matrices, actuators, and transducers. Many of these hydrogels have been thermoresponsive, which undergo a reversible phase separation at the lower critical solution temperature or upper critical solution temperature of the polymer. It has been reported that specifically engineering such hydrogels with additional functionalities, can result in hydrogels responsive to stimuli such as pH, ionic strength, photon flux, and biomolecular binding events. These additional stimuli-responsive characteristics make them useful for numerous applications, such as controlled drug release, tissue regeneration, surface patterning, microfluidic flow control, tunable optics, molecular switches, sensing transducers, and biological assays (bioassays).

Bioresponsive soft materials, which undergo structural and/or morphological changes in response to a biological stimulus, have been investigated for the aforementioned applications, especially with respect to bioassays/biosensors and biomimetic systems. Simple stimuli-sensitive hydrogels have been of interest in a number of fields due to the ability to use external stimuli such as temperature, pH, and photon flux to induce physicochemical changes in the material. More complex hydrogels that are bioresponsive have been engineered by varying the polymer composition, polymeric structure, and the display of specific functional groups. While these materials have been successfully employed for various bio-applications such as controlled drug delivery systems and tissue engineering, they are still of enormous interest for developing more sophisticated materials that display more complex responsivities. One potential application of such bioresponsive hydrogels is biomolecular sensing, where a physicochemical change of a hydrogel is monitored and related to a protein, oligonucleotide, or ligand binding event.

Regarding the development of biological assays, achieving high selectivity to target molecules is preferably accompanied by simplicity in fabrication and ease of use. An inexpensive assay technique that is applicable to a wide range of different affinity pairs with high selectivity would increase the potential for the use of the technique in many applications, such as protein assays, drug screening, chemical sensing, and the detection of genetic defects, such as single nucleotide polymorphisms. Unfortunately, this is not true for all hydrogel-based systems.

For example, U.S. Pat. No. 6,514,689 to Han et al. (i.e., the '689 patent), discloses a hydrogel biosensor confined to a rigid and biocompatible enclosure. The '689 patent further discloses that the hydrogel biosensor measures osmotic pressure within a hydrogel having pendant charged moieties, analyte molecules, and analyte binding partner molecules immobilized within. To achieve meaningful results, however, the hydrogel disclosed in the '689 patent must first be calibrated with a solution of known analyte concentration.

Another example is U.S. Pat. No. 7,045,366 to Huang et al. (i.e., the '366 patent). The '366 patent discloses photo-cross-linked hydrogel blend surface coatings made of cross-linked polysaccharide polymers. The preferred application of the hydrogel blend surface coatings of the '366 patent is for mass spectral analysis of proteins.

A multitude of binding proteins exist for a variety of ligands such as sugars, amino acids, peptides, and inorganic ions. Likewise, enzymes are another class of proteins that can undergo conformational changes as they catalyze a specific reaction. Enzymes can serve as biorecognition elements for substrates, inhibitors, and allosteric effectors. These binding proteins and enzymes come from a range of organisms, some of which grow under extreme environmental conditions. These organisms, termed extremophiles, have adapted to prosper at temperatures as high as that of boiling water in thermal vents (hyperthermophiles) or as low as that of icebergs (psychrophiles). Unlike conventional of-the-shelf proteins that come from organisms that grow at 20 to 37° C., and are non-functional at temperatures above or below this range, proteins from extremophiles can perform under severe conditions.

There are a few examples in the literature where proteins have been integrated into materials capable of displaying a significant change in their characteristics in response to a stimulus, such as U.S. Patent Application Publication No. 20050208469 to Daunert et al. (i.e., the '469 publication). The '469 publication discloses stimuli-responsive hydrogel microdomes integrated with genetically-engineered proteins useful for high-throughput screening of pharmaceuticals. Despite its application in high-throughput screening, manufacturing the hydrogel of the '469 publication requires proteins as an integral part of the polymer matrix. The protein must be genetically engineered to be used as a matrix component and thereafter is mixed homogenously throughout the matrix. In the context of high-throughput screening, this hydrogel requires full diffusion of the analyte through the entire matrix to be fully functional and to yield experimental results after several minutes of analyte exposure.

Thus, there is a need for an inexpensive, highly selective bioresponsive hydrogel that is applicable to a wide range of ligands. In addition, there is a need for a bioresponsive hydrogel that exhibits reversible biosensing characteristics and does not rely on extrinsic labels for detection, quantification, or amplification.

### BRIEF SUMMARY OF THE INVENTION

The present invention is a structure capable of detecting a target molecule including a functionalized polymer matrix having chemically reactive groups and at least one biomolecule attached to the chemically reactive groups to form a surface-modified bioresponsive polymer matrix.

The target detectable by the present invention can include, among others, a sugar, a protein, a nucleic acid, a hormone, a vitamin, a co-factor, or an ion.

The functionalized polymer matrix of the present invention can include natural and synthetic polymers. The functionalized polymer matrix can be functionalized to display chemically reactive groups using a functionalizing agent, which can involve a photo-affinity labeling compound(s), for example, but not limited to, compounds of benzophenones, aryl azides, and diazirines.

The chemically active groups of the present invention can include, among others, phosphoryls, amines, acetates, carboxylates, aldehydes, hydrazides, sulfhydryls, hydroxyls, or ketones.

The biomolecule of the present invention can include, among others, proteins, polypeptides, or nucleic acid molecules. In another aspect of this invention, the structure includes at least two biomolecules forming a biointeractive complex, where the biointeractive complex includes a biointeractive pair, for example, but not limited to, a protein:protein, protein:ligand, oligonucleotide:oligonucleotide, oligonucleotide:protein, oligonucleotide:ligand, antibody:antigen, enzyme: substrate, or protein:drug, among others.

Another aspect of this invention includes a surface-modified bioresponsive polymer matrix that is a microlens. In another aspect of the invention, the surface-modified bioresponsive polymer matrix is a hydrogel. The hydrogel can further be formed into a microlens.

In another preferred embodiment, the invention is an assay system for detecting an analyte of interest, including polymer particles covalently conjugated with multiple copies of biointeractive pairs, a container for loading the conjugated particles, an analyte in solution, and a detector for detecting analyte disruption of the biointeractive pairs.

The analyte detectable by the assay system can be a sugar, a protein, a nucleic acid, a hormone, a vitamin, a co-factor, or an ion, among others.

The polymer particles of the invention include polymer particles in the form of a dried powder or a swollen gel.

The biointeractive pairs covalently conjugated to the polymer particles can be, for example, but not limited to, protein:protein, protein:ligand, oligonucleotide:oligonucleotide, oligonucleotide:protein, oligonucleotide:ligand, antibody:antigen, enzyme:substrate, or protein:drug pairs, among others. Furthermore, the biointeractive pairs can interact via non-covalent bonds including ionic bonds, hydrogen bonds, hydrophobic interactions and van der Waals forces.

The invention can further include a container such as a pipette tip, a micropipette tip, a plate well, or a centrifuge tube, among others.

Analyte disruption of the biointeractive pairs can occur via non-covalent binding of the analyte to one of the biointeractive molecules. Consequently, the detector will detect the analyte disruption of the biointeractive pairs via, for example, but not limited to, controlled disassembly of colloidal gels, visualization of microgel-based microlenses, or particle counting following substrate deposition.

In another aspect, the assay includes a hydrogel formed from the polymer particles.

In yet another aspect of the invention, the assay system is portable.

The invention is also a method of making a synthetic matrix capable of detecting a target, comprising polymerizing polymer particles, functionalizing the polymerized particles using a functionalizing agent to produce chemically active groups, and attaching one or more biomolecules to the functionalized particles wherein the one or more biomolecules noncovalently bind the target molecule.

The polymerized polymer molecules include, for example, acrylates, acrylamides, acetates, acrylic acids, vinyl alcohols, and glycols, among others.

The functionalizing agent used with the invention to produce chemically active groups can be a photo-affinity labeling compound, including but not limited to, compounds comprised of benzophenones, aryl azides, and diazirines. Examples of chemically active groups including but not limited to, phosphoryls, amines, acetates, carboxylates, aldehydes, hydrazides, sulfhydryls, hydroxyls, or ketones, among others.

The method of the invention can include covalently attaching one or more biomolecules to the functionalized particles. The one or more biomolecules can further form a non-covalently bound biointeractive complex, which can further comprise a biointeractive pair. Biointeractive pairs can be, for example, but not limited to, protein:protein, protein:ligand, oligonucleotide:oligonucleotide, oligonucleotide:protein, oligonucleotide:ligand, antibody:antigen, enzyme:substrate, or protein:drug pairs, among others.

The target molecule then can disrupt the biointeractive pair(s) and induce a conformational change in the synthetic matrix. The conformational change can be detected optically or non-optically.

In another aspect, the method of the invention includes forming a hydrogel from the polymerized polymer molecules. A microlens can be formed by placing either the polymerized polymer particles or the hydrogel onto a substrate.

The invention is also a portable device for measuring the concentration of an analyte, comprising a functionalized polymer matrix having chemically reactive groups, at least one biomolecule attached to the chemically reactive groups to form a surface-modified bioresponsive polymer matrix, a container for holding the surface-modified bioresponsive polymer matrix, and a detection means for detecting a change in the bioresponsive polymer matrix in response to the analyte.

The analyte can be, for example, a sugar, a protein, a nucleic acid, a hormone, a vitamin, a co-factor, or an ion, among others.

The functionalized polymer matrix includes polymer particles covalently conjugated with multiple copies of biointeractive pairs. The polymer particles can be in the form of a dried powder or a swollen gel. The biointeractive pairs can be protein:protein, protein:ligand, oligonucleotide:oligonucleotide, oligonucleotide: protein, oligonucleotide:ligand, antibody:antigen, enzyme:substrate, or protein:drug pairs. The biointeractive pairs can interact via non-covalent bonds, such as ionic bonds, hydrogen bonds, hydrophobic interactions and van der Waals forces.

The container of the invention can be a pipette tip, a micropipette tip, a plate well, or a centrifuge tube.

The detector of the invention can detect analyte disruption of biointeractive pairs via controlled disassembly of colloidal gels, visualization of microgel-based microlenses, or particle counting following substrate deposition.

In another aspect, the portable device includes a hydrogel formed from the polymer particles.

In addition to these preferred embodiments, the invention further comprises a detection system capable of delivering qualitative results in “real time” and quantitative results in near “real-time.” That is, the surface-based nature of the invention coupled with the sensitivity and specificity of biomolecule augmentation allows for superior results with respect to reaction time and experimental certainty. For the purpose of the invention, “real-time” can be about 30 seconds or less and near “real-time” can be about one minute or less.

### BRIEF DESCRIPTION OF THE FIGURES

FIGS. 1a-d are diagrams of the hydrogel microlens assay.

FIGS. 2a-b are images of the dependence of microlens swelling as a function of avidin concentration.

FIGS. 3a-d depict fluorescence microscopy images of hydrogel microlenses.

FIGS. 4a-d depict the sensitivity of the hydrogel microlens assay to the number of the active binding sites on avidin.

FIGS. 5a-c show the effects of the monovalent binding and the nonspecific adsorption of bare microgels and the biotinylated microgels.

FIGS. 6a-b depict the influence of a polyclonal anti-biotin antibody on the hydrogel.

FIGS. 7a-d illustrate the reversibility of the hydrogel microlens assay.

FIG. 8 is a diagram representing the inverted light microscopy setup used for aqueous phase imaging experiments.

FIGS. 9a-d are a diagram of label-free biosensing using bioresponsive hydrogel microlenses.

FIGS. 10a-e show the influence of polyclonal anti-biotin antibody concentration on lensing and the optical model of lens structure after photo-irradiation.

FIGS. 11a-f demonstrate the reversibility of the bioresponsive microlenses as shown by the projection of the square pattern.

FIGS. 12a-c show the effects of nonspecific adsorption on the optical properties of bioresponsive microlenses.

FIGS. 13a-h show tuning microlens sensitivity.

### DETAILED DESCRIPTION OF PREFERRED EMBODIMENTS

A preferred embodiment of the present invention is a structure capable of detecting a target molecule, comprising a functionalized polymer matrix having chemically reactive groups and at least one biomolecule attached to the chemically reactive groups to form a surface-modified bioresponsive polymer matrix.

As used herein, a functionalized polymer matrix is a synthetic or naturally-occurring polymer that is functionalized to display chemically reactive groups. A functionalized polymer is a polymer that is modified to make it useful for a given application. Modifications include chemical or physical modifications that allow, for example, but not limited to, molecule-to-polymer interactions, surface-to-surface interactions, end-group protection, end-group deprotection, or photo-tethering.

Examples of biomolecules include proteins, polypeptides, or nucleic acid molecules, including aptamers, among others. A surface-modified bioresponsive polymer matrix is a polymer matrix having biomolecules on or near the surface of the polymer matrix and not integrated throughout the matrix. The biomolecules can be specific for binding one or more target molecules.

Aptamers are single-stranded DNA or RNA molecules that bind with high affinity to specific target or analyte molecules. Such analyte molecules can be drugs, vitamins, hormones, antibodies, enzymes, co-factors, nucleotides, and proteins, among others. Aptamers can range from between approximately eight (8) to 120 or more nucleotides in length. Contained within this nucleotide sequence is a minimal sequence needed for binding to the analyte. Such sequence is normally between approximately 15 to 50 nucleotides in length. Aptamers undergo a conformational change after binding specific analytes. The binding constant of aptamers to their specific analyte molecules ranges from micromolar to sub-nanomolar ranges. Aptamers have a number of advantages over other molecules that specifically bind target molecules, including, for example, but not limited to, specificity and ease of synthesis.

Upon binding a target molecule, a detectable signal occurs in the polymer matrix. Examples of such detectable signals are fluorescent signals, optical signals, electrochemical signals, pressure changes, changes in dielectric constant, mass changes, volume changes, and temperature changes, among others. Biomolecules can be used as a sensor, particularly within a micromechanical or nanomechanical device or biosensor to detect the presence of the analyte and to generate a signal, which is transmitted to a transducer.

The target can be, for example, but not limited to, an analyte or biochemical, such as that found in an organism (e.g., bacteria, yeast, animals, humans, plants, among others), a sugar, a protein, a nucleic acid, a hormone, a vitamin, or a co-factor, among others. The target molecule can also be an ion, such as a hydrogen ion, a hydroxyl ion, an oxyanion (e.g., phosphate, sulfate, among others) or a cation (e.g., calcium ion, among others), among others. The bonds that form between the target and the biomolecule include all chemical bonds except covalent bonds. Examples of such chemical bonds are ionic bonds, hydrogen bonds, hydrophobic interactions and van der Waals forces, among others. Further, the target can be molecules such as drugs, vitamins, hormones, antibodies, enzymes, co-factors, nucleotides, proteins, among others.

In another embodiment, the invention is an assay system for detecting an analyte of interest, comprising polymer particles covalently conjugated with multiple copies of biointeraction pairs, a container for loading the conjugated particles, an analyte in solution, and a detector for detecting analyte disruption of the biointeraction pairs.

For example, but not limited to, the polymer matrix surface can be covalently conjugated with multiple copies of biointeractive pairs (e.g., protein:protein, protein:ligand, oligonucleotide:oligonucleotide, oligonucleotide:protein, oligonucleotide:ligand, antibody:antigen, enzyme: substrate, protein:drug, among others). These pairs will form inter-particle and/or intra-particle cross-links. The bioconjugated polymer matrix particles can be in the form of a dried powder or a swollen gel for loading into a sampling or incubation container. This container can be one of a variety of disposable components suitable for bioassay protocols (e.g., a pipette or micropipette tip, a centrifuge tube, a plate well, or the like).

The assay would take advantage of, for example, but not limited to, one or more of the following readout mechanisms: controlled disassembly of colloidal gels, visualization of microgel-based microlenses, or particle counting following substrate deposition. The assay will rely on the disruption of biomolecule-based, non-covalent, inter- and/or intra-particle cross-links. These cross-links will be disrupted by the analyte or target molecule of interest, causing a measurable change in one or more of the readout mechanisms.

Further by way of example, another preferred embodiment of the assay can include a micropipette tip (or a reaction and sampling container), which is pre-loaded with a polymer matrix that is surface-modified for a specific target analyte. In the case of a micropipette tip, the tip would be used to withdraw a controlled volume of the sample of interest. The sample would be allowed to incubate in the container for a predetermined period of time, after which the sample would be expelled onto a substrate (e.g. microscope slide, microscope coverslip, or the like). This can be followed by drying or rinsing steps, depending on the assay to be performed. One or more of the following readout mechanisms would then be used to determine the analyte concentration. First, the microlens focal length changes will increase with analyte or target molecule concentration in the event that intra-particle cross-links are disrupted. Second, the degree of particle aggregation and/or the number of particles expelled onto the surface will change with analyte concentration in the event that inter-particle cross-links are disrupted. Third, a combination of the two effects can be used to correlate the disruption of both inter- and intra-particle cross-links.

Particles possessing multiple different sensitivities to the target analyte can be used to broaden the dynamic range or specificity of the assay. Particles that are not responsive to the analyte can be included to act as internal standards for microlens focal length, thereby providing for relative instead of absolute measurements of focal length.

In another preferred embodiment, the invention is further a method of making a matrix capable of detecting a target molecule, comprising polymerizing polymer particles, functionalizing the polymerized particles using a functionalizing agent, and attaching one or more biomolecules to the functionalized particles. Preferably, the one or more biomolecules noncovalently bind the target molecule such that the binding is more easily reversed and the matrix can be reused.

In a preferred embodiment, the matrix is a hydrogel. Hydrogels, which can further be formed into microlenses, can exhibit dramatic effects of swelling or shrinking upon a stimulus. One such stimulus is movement or conformational change of biomolecules attached thereto. Another type of stimulus occurs when there is a change in pH in the environment in which the hydrogel is present. Such local pH change causes water and counter-ions to move in or out of the hydrogel and this induces swelling or shrinking of the hydrogel. Certain types of hydrogels undergo abrupt changes in volume in response to changes in pH, temperature, electric fields, saccharides, antigens and solvent composition, among others. Natural and artificial hydrogels can also be forced to shrink or swell by applying a bias on a metal electrode underneath or embedded in a hydrogel gel.

A hydrogel can be prepared using many suitable monomers that, when polymerized, forms a hydrogel. Such monomers include, but are not limited to, acrylates, acrylamides, acetates, acrylic acids, vinyl alcohols, and glycols.

The following non-limiting examples illustrate the composition and method of making the instant invention.

### EXAMPLE 1

A hydrogel microlens was developed in which specific protein binding events were monitored as changes in the microlens focal length via brightfield optical microscopy. The microlens focal length can be tuned by multivalent protein binding, where the protein:ligand association formed a cross-linker in the hydrogel network. In this work, a microlens having a tunable focal length was developed by coupling an antigen:antibody pair directly to the microlens, thereby providing for a reversibly switchable cross-link on the microlens. This forms the basis of a new biosensing construct that is reversible, and simultaneously acts as the biosensor scaffolding/immobilization architecture, transducer, and amplifier, while providing for broad tunability of the analyte concentration to which the microlens is sensitive. Furthermore, this construct is exceedingly resistant to spurious signals due to non-specific binding, since the microlens bioresponsivity is dependent on the reversible displacement of protein:ligand interactions.

Hydrogel microparticles (about one micron in diameter) composed of poly(N-isopropylacrylamide-co-acrylic acid) (pNIPAm-co-AAc) were fabricated via aqueous free-radical precipitation polymerization. Manufacture of hydrogel particles of this nature is known to those of ordinary skill in the art, and it is well-recognized in the art that hydrogels can be manufactured using different protocols and components to suit different applications. For example, U.S. Pat. No. 4,871,490 to Rosiak et al., discloses a method for manufacturing hydrogels for use as dressings. By way of further example, U.S. Pat. No. 7,001,987 to Van Dyke discloses a method for making a hydrogel with controllable mechanical, chemical, and biological properties.

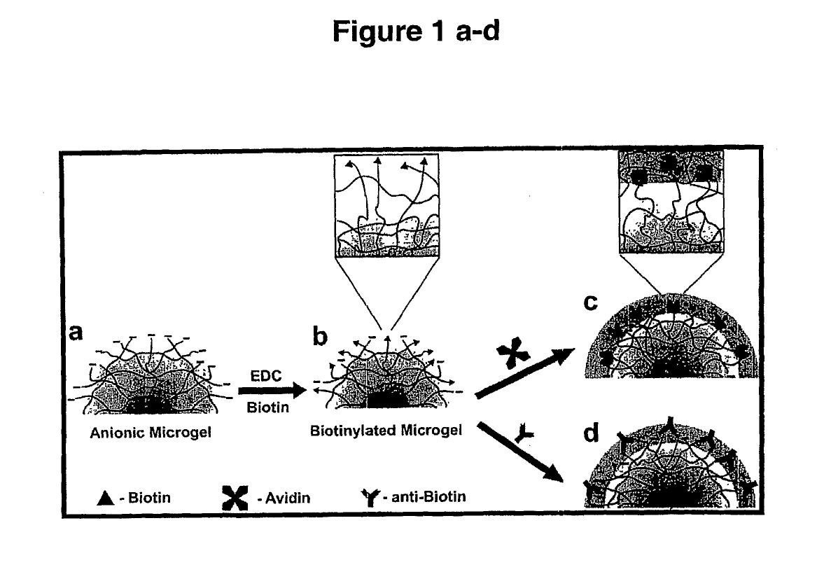

It has been previously demonstrated that similar particles can be used to create self-assembled arrays of hydrogel microlenses on solid supports. To render the microgels antibody reactive, a portion of the AAc groups are used to couple an antigen (biotin, as H₂N-Biotin) via 1-Ethyl-3-(3-dimethylaminopropyl)-carbodiimide (EDC) coupling (FIG. 1b). Those of ordinary skill in the art will recognize and appreciate that functionalization can be accomplished using many classes of labeling compounds, such as but limited to, photo-affinity compounds. Classes of photo-affinity labeling compounds include, for example, but not limited to, benzophenones, aryl azides, and diazirines. In addition, those of ordinary skill in the art will further recognize and appreciate that various types of chemical groups can be activated by functionalization. These groups include, for example, but not limited to, amines, carboxylates, aldehydes, hydrazides, sulfhydryls, hydroxyls, and ketones.

The biotin-functionalized microgels can then be Coulombically assembled onto a 3-aminopropyltrimethoxysilane (APTMS)-prepared glass substrate to form supported microlenses. Bioresponsive microlenses are then prepared by exposure to a buffered solution of polyclonal anti-biotin, which binds to the microlenses via antibody:antigen interactions. See FIG. 1d.

This example utilizes the biotinylated pNIPAm-co-AAc hydrogel microparticles as both the protein recognizing and transducing material. In this strategy, a portion (approximately 50%) of the acid groups of the microgels are conjugated to the biotin ligand via EDC coupling. See FIGS. 1a-b. These biotinylated microgels then interact with multivalent proteins (avidin or anti-biotin), which form additional cross-links between polymer chains in the network. Such a cross-linking event results in the change in the equilibrium swelling volume of the microgel and hence an increase in the local refractive index (RI) of the microgel. The optical properties of the hydrogel microlenses are dependent on the RI contrast between the hydrogel and the surrounding medium. Also, microlenses formed from pH and temperature responsive gels are able to project images of different fidelities in response to pH and temperature changes, respectively.

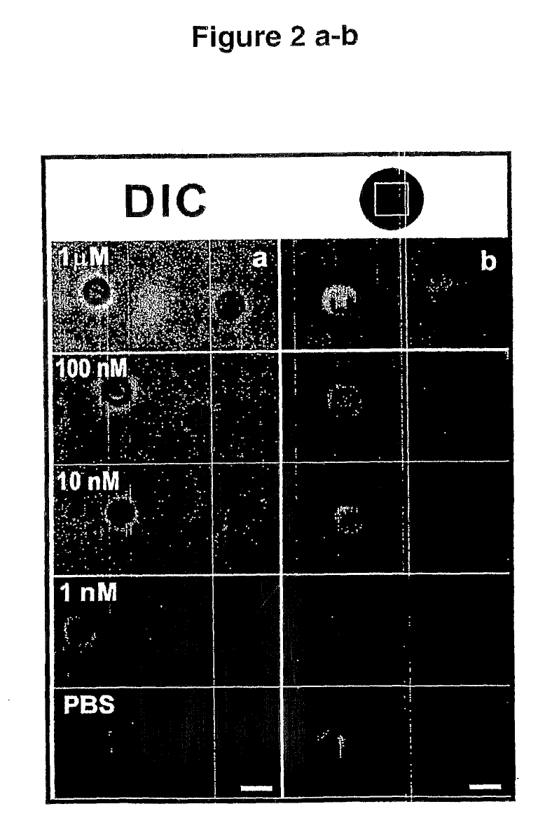

Substrates containing a random binary distribution of microlenses were prepared to investigate the potential utility of hydrogel microlenses in a protein assay system where both pNIPAm-co-AAc microlenses and biotinylated pNIPAm-co-AAc microlenses are present in approximately equal number densities. The microlenses were then exposed to various concentrations of avidin solutions (FIG. 1c) by introduction of 150 μL of the proper solution into the void space of a microlens array/silicone gasket/coverslip sandwich assembly. The effects of avidin concentration on the optical properties of the microlenses are shown in FIGS. 2a-b (white horizontal bar in this and other figures represents a length of two micrometers). Only the biotinylated hydrogel microlenses (left elements in each panel) show a difference in appearance in the differential interference contrast (DIC) images as the avidin concentration is increased, with the most marked difference being the formation of the dark circle at the particle periphery (FIG. 2a, column). The non-biotinylated microlenses (right elements in each panel) do not show any apparent change at different concentrations of avidin. The biotinylated microlenses exhibit a large change in image formation (white square) at 100 nM avidin (equivalent to 15 pmoles of protein), while the non-biotinylated hydrogel microlenses show a weak, poorly focused image over the entire range of the avidin concentrations. See FIG. 2b, column. The change in lens projection observed for the biotinylated lenses appears to be the formation of a double image, where the periphery of the particle appears bright, while a small, more tightly focused square appears at the center of the microlens. These phenomena are due to the local RI change of the biotinylated hydrogels caused by the formation of biotin-avidin networking on the surface at a critical avidin concentration. The higher RI decreases the effective focal length of the microlens, hence creating a smaller, more tightly focused image of the white square pattern. It can also be the case that the higher RI at the microlens surface causes an increase in light scattering, which can be the origin of the bright appearance of the particle periphery. Regardless of the detailed origins of the image formation, it is clear that the ligand-modified lenses are sensitive to protein binding, and directly report on that binding through a change in both microlens appearance (FIG. 2a, column) and microlens performance (FIG. 2b, column). Further, note that the biotinylated hydrogel microlenses have very different optical properties (focal lengths) than the non-biotinylated microlenses before introduction of the protein (e.g. in phosphate buffered saline (PBS) only). This arises from the decrease in the number of acidic sites in the biotinylated microgels, which decreases the equilibrium swelling volume and hence increases the RI of the microlens.

### EXAMPLE 2

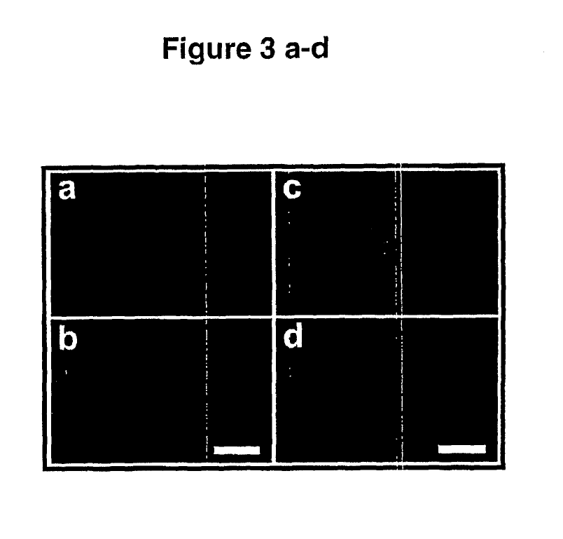

In designing many affinity-based assay systems, mediation of non-specific adsorption and enhancement of selectivity are two of the key figures of merit. Thus, fluorescence microscopy was used to investigate specific biotin-avidin binding to the hydrogel microlenses (FIGS. 3a-b). Note that the avidin and hydrogels are labeled by fluorescent chromophores with red (Texas red) and green (fluorescein) emission spectra, respectively. The appearance of the red fluorescence at the periphery of the left element in FIG. 3a confirms that the avidin only binds to the surface of the biotinylated hydrogel microlens. There is no discernable non-specific adsorption to the non-biotinylated hydrogel microlens at the same solution avidin concentration (right element in FIG. 3a). Note that in PBS buffer, both microlenses display only green fluorescence due to fluorescein, although the biotinylated microlens appears to have weaker fluorescence intensity than that of the non-biotinylated one. See FIG. 3b. This can be due to a difference in the photo-bleaching rate between the two particles, or it can be that biotin acts as a quencher when placed in close proximity to fluorescein. Finally, brightfield transmission microscopy was used to scrutinize the selectivity of these assay system by exposing the biotinylated microlenses to a solution of anti-avidin (FIGS. 3c-d). The DIC images of the hydrogel microlenses are unchanged by the presence of anti-avidin at a solution concentration of 730 nM (equivalent to 110 pmoles, FIG. 3c) as compared to images acquired in PBS buffer solution (FIG. 3d).

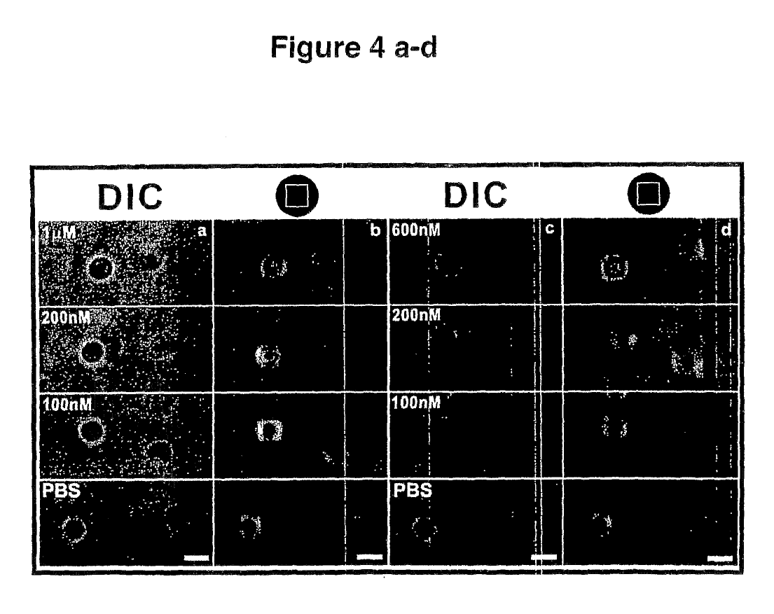

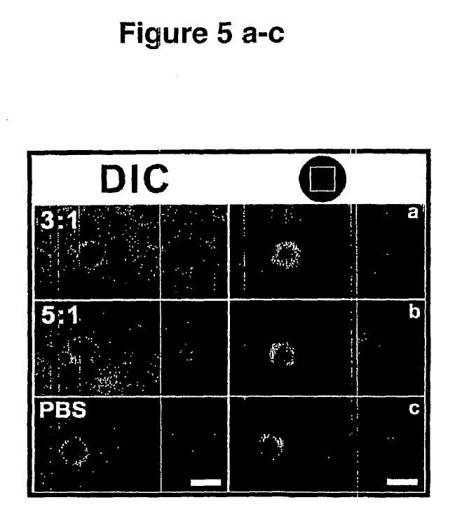

As described above, the avidin concentration dependent microlens response could be caused by an increase in the network cross-link density, which is due to the ability of avidin to bind up to four equivalents of biotin. In order to prove the requirement of multivalent binding, the sensitivity of the microlens assay to avidin that had been equilibrated with different amounts of free biotin was investigated. FIG. 4 shows the DIC (FIGS. 4a and 4c, columns) and lens projection (FIGS. 4b and 4d, columns) images of the hydrogel microlenses exposed to avidin solutions pre-equilibrated with one equivalent (FIGS. 4a and 4b, columns) or two equivalents (FIGS. 4c and 4d, columns) of biotin. Because of the extraordinarily low dissociation constant (and hence small dissociation rate constant) of the biotin:avidin pair (K_{d}˜10^{-13 to -15}) the free biotin is not expected to exchange with the hydrogel-bound biotin on the timescale of the experiments. Thus, this experiment allows for a measure of avidin-based cross-linking as a function of the number of free binding sites. Comparing the collective data in FIG. 4 with that in FIG. 2, where free avidin is used, suggests that the hydrogel microlens is less sensitive to avidin that has been pre-equilibrated with biotin than with free avidin. In the case of 1:1 ratio of biotin:avidin, the microlens is observed to “turn on” at approximately 200 nM (30 pmoles of avidin), while for 2:1 biotin:avidin, the lens is not switched until approximately 600 nM (90 pmoles of avidin). This behavior can be reasonably understood by the fact that the free avidin statistically will have more opportunities to form cross-links than avidin with partially occupied binding sites. To eliminate the possibility that the focal length change is caused by monovalent binding and/or nonspecific binding of avidin, microlens arrays were exposed to solutions of avidin pre-equilibrated with three (monovalent avidin) and five (excess biotin) equivalents of biotin. See FIGS. 5a-c. Under these conditions, no discernable change was observed in lens focal length due to the statistical improbability of protein-based cross-linking under conditions where one or fewer binding sites is available on the protein.

### EXAMPLE 3

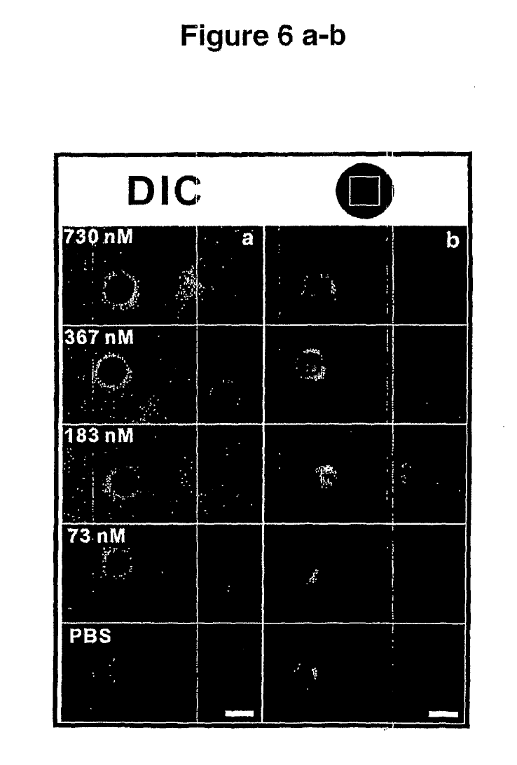

A weaker binding protein-ligand interaction was investigated to evaluate the generality of the assay for protein detection. In this case, a polyclonal antiserum (immunoglobulin G, or IgG fraction) raised in goat against biotin was used as the cross-linking protein. Note that an IgG is different from avidin in a number of ways. First, IgG has a higher molecular weight (approximately 150 kDa vs. approximately 66 kDa for avidin). Second, IgG has only two (2) binding sites (paratopes) for biotin. Third, IgG is expected to have a much higher dissociation constant than avidin. Typical effective (ensemble) dissociation constants for polyclonal antisera are on the order of K_{d}˜10⁻⁶ to 10⁻⁹ M. FIG. 6a (column) shows the DIC images and FIG. 6b (column) shows the projected pattern images as a function of anti-biotin concentration, respectively. The hydrogel microlens assay displays a focal length change at a concentration above approximately 367 nM (equivalent to 55 pmoles), with the general microlens appearance being very similar to that observed for avidin binding. Comparing FIGS. 4c and 4d (columns) with the columns of FIGS. 6a and 6b (where the effective number of binding sites to biotin is same but the K_{d} values are different), the microlens assay is more sensitive to anti-biotin than it is to avidin, despite avidin's lower K_{d} value. While it is possible that this arises from the higher molecular weight of the IgG, it is also reasonable to consider the larger distance between binding sites in the IgG. It can be the case that the IgG is statistically a better cross-linker simply because it can access more biotin than the smaller avidin. Furthermore, the anti-biotin assay is less sensitive than the avidin assay used to produce the data in FIGS. 4a and 4b, where the avidin has three active binding sites. Thus, the sensitivity of the cross-linking assay will be due to the protein:ligand affinity, the number of ligand binding sites, and the distance between binding sites on the protein.

### EXAMPLE 4

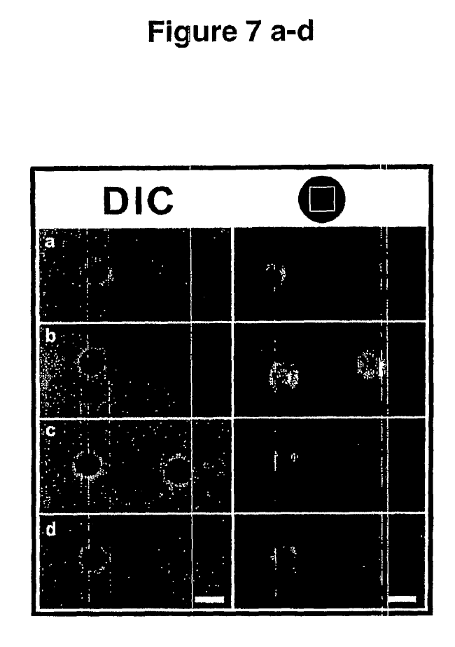

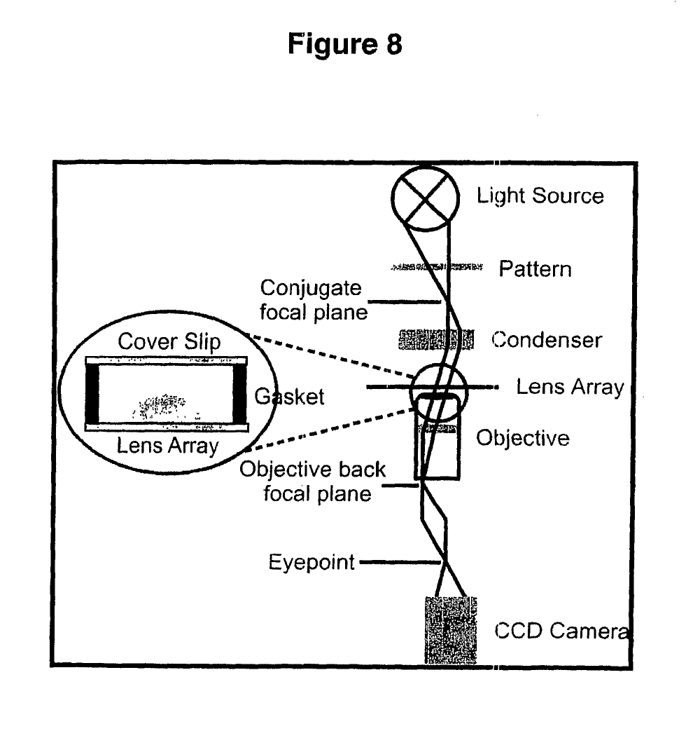

The reversibility of the hydrogel microlens assay is shown in FIGS. 7a-d. The hydrogel microlenses were stepwise exposed to PBS buffer (FIG. 7a), polyclonal anti-biotin solution (FIG. 7b), and biotin solution (FIGS. 7c-d). When the antibody-bound microlenses are exposed to a solution of the free ligand, the focal length of the microlens reverts back to its original state, suggesting that the protein-based cross-links have been disrupted (FIGS. 7c-d). This result suggests that this construct can be used in a displacement-type assay. For example, but not limited to, each microlens can contain both a tethered protein and a tethered ligand, where association between the two results in a cross-linking point and hence a decrease in focal length. However, upon exposure to the free ligand or protein (depending on what is to be assayed), these cross-links would be disrupted, thereby increasing the lens focal length, which can be visualized on a simple optical microscope. A representative example of such an experimental setup appears in FIG. 8. A displacement assay of this type would have the advantage of being reversible, since the displaced moiety would remain tethered to the microlens. Following washing, the protein:ligand cross-link would be re-formed, thereby resetting the microlens in the “on” state. Furthermore, if one were able to tune either the dissociation constant of the tethered protein:ligand pair, or the critical number of cross-links required for microlens modulation, the sensitivity of the assay to the solution concentration of analyte can be tuned to a level appropriate for a particular application.

### EXAMPLE 5

To demonstrate label-free biosensing using hydrogels, hydrogel microparticles (about one (1) micron in diameter) were fabricated according to EXAMPLE 1 with an additional functionalization step and by adding a photo-ligation step. Those of ordinary skill in the art will recognize and appreciate that functionalization can be accomplished using many classes of labeling compounds, such as but not limited to, photo-affinity compounds. Classes of photo-affinity labeling compounds include, for example, but not limited to, benzophenones, aryl azides, and diazirines. In addition, those of ordinary skill in the art will further recognize and appreciate that various types of chemical groups can be activated by functionalization. These groups include, for example, amines, carboxylates, aldehydes, hydrazides, sulfhydryls, hydroxyls, and ketones, among others.

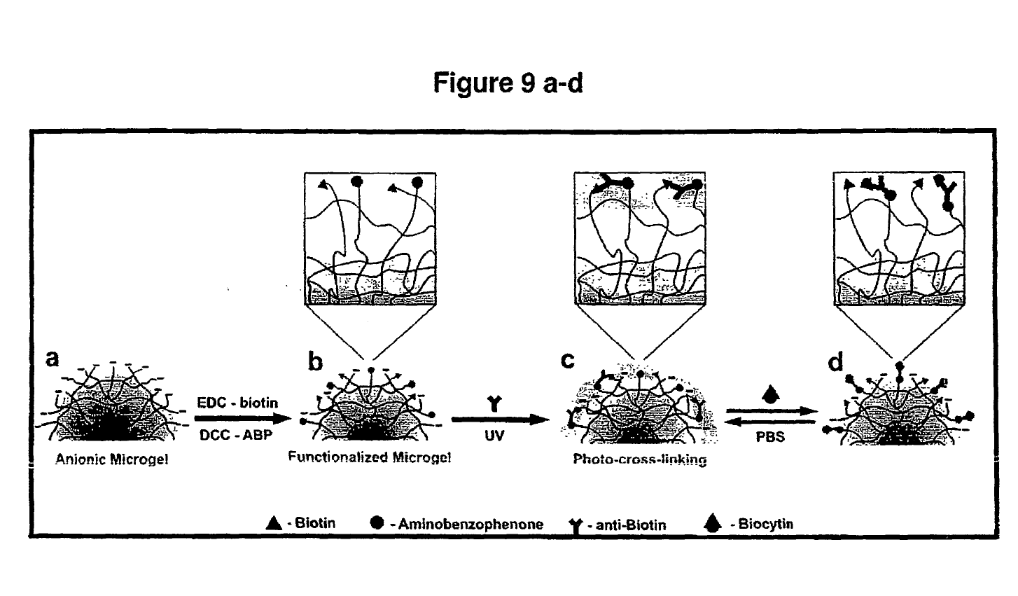

In addition to the EDC coupling used in EXAMPLE 1, a different portion of chemical groups were functionalized using aminobenzophenone (ABP) via dicyclohexylcarbodiimide (DCC). See FIGS. 9a-b. Functionalization by ABP allows for photo-tethering of anti-biotin after it is associated to the microlens via native antibody:antigen association. Photo-ligation of the surface-tethered ABP to the antigen-bound antibody is accomplished via ultraviolet (UV) irradiation (FIG. 9c). Thus, the microlens surface is decorated with multiple antibody:antigen-based cross-links, which can be disrupted by introduction of free antigen to the surrounding medium (FIG. 9d). Since the antibody is covalently tethered to the microlens surface, washing with antigen-free media results in re-assembly of the tethered antibody:antigen pairs, thereby providing for a reversible biosensing microlens.

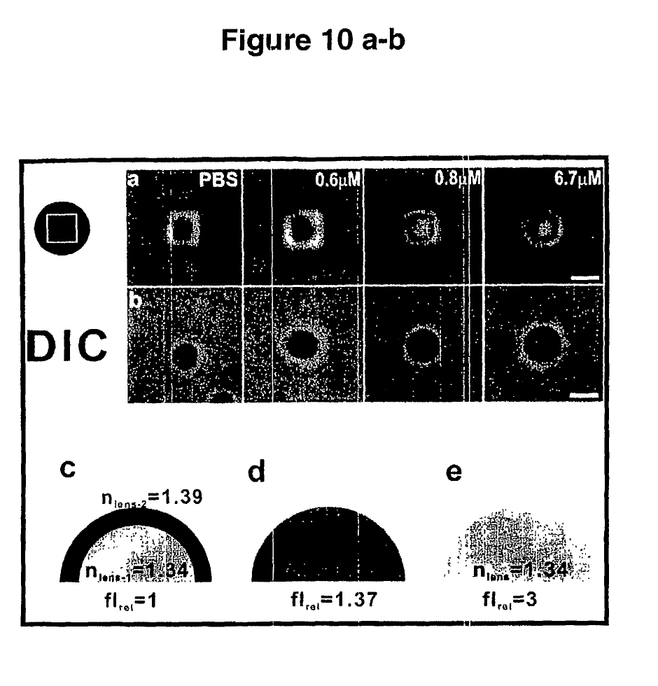

The behavior of microlenses formed following incubation with different concentrations of polyclonal anti-biotin is shown in FIGS. 10a and 10b. Under these conditions, microlenses show antibody concentration dependence in the DIC images with the formation of a dark circle at the particle periphery (FIG. 10b). FIGS. 10a and 10b further show changes in the image projection through the microlenses. A schematic of the microscope setup is shown in FIG. 8. Above a critical antibody concentration, the lens is in the “on” configuration, while below that concentration the lens is “off”.

These optical effects are due to the local RI change of the hydrogel microlenses caused by the formation of biotin:anti-biotin based cross-links at the microlens surface. The critical anti-biotin concentration represents the point at which the number of cross-linking points is sufficient to cause the microgel periphery to deswell. Below that concentration, the elastic restoring force of the hydrogel network exceeds the free energy change associated with multivalent antibody binding. In this fashion, the intrinsic binding affinity of the antibody:antigen pair is modulated by the negative entropy associated with gel restriction.

### EXAMPLE 6

To illustrate the effect of microlens surface deswelling on the overall optical properties, a series of 2-D optical raytracing simulations (Raytrace v. 2.18, see FIGS. 10c-10e) were performed in a medium with n=1.33 (RI of water). In the lens “on” state (FIG. 10c), the microlens is modeled as meniscus+plano-convex compound lens with slightly higher RI at the periphery (n=1.39 vs. 1.34 in the bulk) due to a surface localized binding of biotin:anti-biotin. The simulations show that the compound lens structure produces a significantly shorter relative focal length (flᵣₑₗ=1.00) as compared to the unmodified hydrogel microlens modeled as a uniform plano-convex lens (n=1.34; flᵣₑₗ=3.00; see FIG. 10e).

In this figure, the surface localized binding case was compared to a plano-convex lens where the increase in the RI due to antibody:antigen binding is uniformly distributed over the entire optic (n=1.35; flᵣₑₗ=1.37; see FIG. 10d). In light of the simulation results, it is clear that by limiting the hydrogel responsivity to the particle periphery, one can potentially have a higher sensitivity to binding events due to a concomitant localization of the RI changes. Furthermore, we expect that these structures should display fast response times due to the short mass transport distance required to elicit an optical response.

### EXAMPLE 7

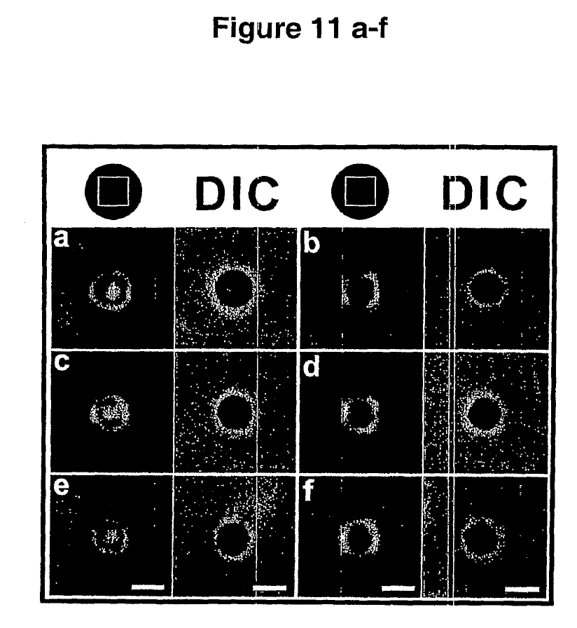

The lens bioresponsivity is highly reversible, as shown in FIGS. 11a-f. Reversibility allows the invention to be used more than once. That is, for example, once the surface-modified bioresponsive polymer matrix binds its specific analyte and, for example, but not limited to, causes swelling or shrinking of a hydrogel, it would be advantageous if the surface-modified bioresponsive polymer matrix can be returned to its original state, for example, but not limited to, the state in which no analyte is bound by the surface-modified bioresponsive polymer matrix.

In this example, the biotin-ABP-functionalized hydrogel microlenses were incubated with a 6.7 μM solution (equivalent to 670 pmol) of polyclonal anti-biotin, followed by UV irradiation to covalently tether the antigen-associated antibodies to the microlens. The changes in the microlens-projected image were then monitored during alternating exposure to 10 mM PBS (FIGS. 11a, 11c, 11e, projected square and DIC columns) and one (1) mM biocytin (FIGS. 11b, 11d, and 11f, projected square and DIC columns) solutions. Biocytin is a water-soluble analogue of biotin. The microlens is initially observed be in the “on” state in PBS buffer, which we characterize as the formation of a double square image in image projection mode and the dark circle at the particle periphery in the DIC image. When the microlenses are then exposed to a solution of free biocytin, the microlenses are observed to switch to the “off” state, as characterized by a single square image (projection mode) and the disappearance of the black circle (DIC mode). This change in microlens focal length arises from disruption of the bound antibody:antigen pairs by competitive displacement with free antigens from solution. When the microlens is returned to an antigen-free buffer, the tethered antibody:antigen pairs re-assemble as the free antigens dissociate from the microlens. This response can then be cycled by repeated exposures to either antigen-containing or antigen-free buffer.

These results indicate at least two things. First, the results indicate that the microlens response is thermodynamically reversible, i.e., the initially photo-coupled state is a relatively low energy state. Second, the results indicate that the antibodies are indeed coupled to the microlens, as reversibility would not be expected if the first displacement interaction led to dissolution of antibody from the microlens surface.

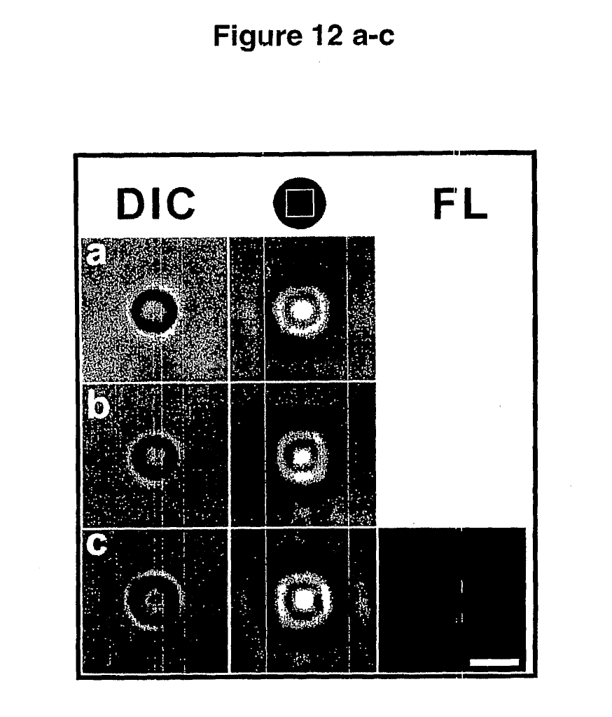

This displacement-based mode of action yields microlenses that are insensitive to non-specific binding, as well as specific binding to non-paratope regions of the tethered antibody. See FIGS. 12a-c; DIC, projected square, and fluorescence (FL) columns. In this experiment, the hydrogel microlenses in the DIC, projected square, and FL columns were prepared with 1 μM anti-biotin. Thereafter, the hydrogel microlenses were exposed to a volume of 150 μL of either 10 mM PBS buffer (pH 7.5) (FIG. 11a), 5.5 μM anti-avidin (FIG. 11b), or 5.5 μM rabbit anti-goat IgG conjugated with Alexa Fluor 594 (FIG. 11c). No detectable change occurs in the hydrogel microlenses under these conditions, indicating insensitivity to nonspecific and noncompetitive binding.

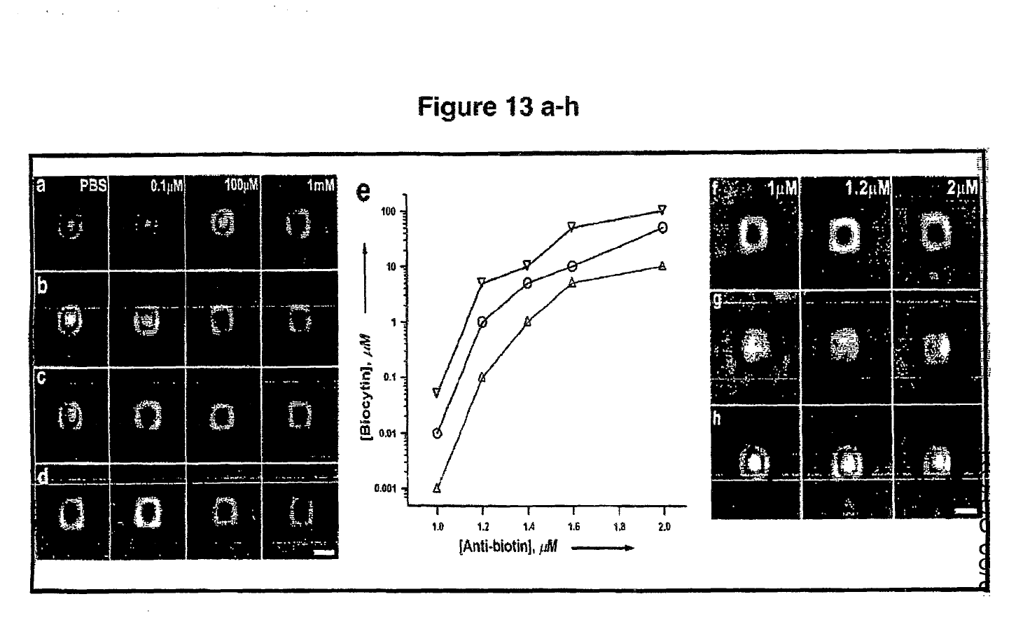

In designing many sensor systems, selectivity, sensitivity, and dynamic range are the key factors to realize a sensor for real world utilization. In the light of above results, we are able to prepare bioresponsive hydrogel microlenses, which have high specificity in a label-free format due to the use of a displacement/competitive binding scheme. The response is essentially binary (on/off), however, which does not allow for quantitative analysis over a wide range of analyte or target molecule concentrations. Therefore, it should be possible to tune the sensitivity of a hydrogel (e.g., a hydrogel microlens) by changing the number of biointeractive pairs (e.g., antibody:antigen cross-links) present on the microlens, and consequently the number that must be displaced to induce a response. This can be accomplished, for example, but not limited to, by changing the concentration of polyclonal anti-biotin used in the photo-cross-linking step to set initial lens “on” state. These data are shown in FIGS. 13a-13h.

The minimum concentration of analyte or target molecule required to switch the hydrogel state from “on” to “off” is inversely dependent on the concentration of biomolecules attached to the hydrogel, given that the biomolecules and the analyte or target molecules specifically bind to each other. For example, the concentration of biocytin required to switch the microlens state is inversely dependent on the concentration of antibody used in the photo-cross-linking step. This can be understood by considering the thermodynamics of the system. In the tethered biointeractive pair system (e.g., an antibody:antigen system), the thermodynamics of hydrogel swelling are intimately coupled with those of the biointeractive pair. That is, the effective affinity of an individual binding pair must be reduced in the case where the gel is deswollen relative to its equilibrium state. Essentially, this is a state where the total free energies of the biointeractive pair binding overcome the required reduction of network entropy necessary for deswelling. Thus, there should be a critical number of cross-linking points that result in observable hydrogel deswelling. If the hydrogel microlenses are prepared with excess binding pairs above that critical point, then the individual hydrogel microlenses will reswell only after a suitably large number of displacement events have occurred. If the number of cross-linking points is just slightly above this critical point, however, only a few displacement events will result in gel swelling.

### EXAMPLE 8

In this example, tuning of the microlens sensitivity is shown. Hydrogel microlenses were prepared as in EXAMPLE 5 but using different concentrations of anti-biotin before photo-cross-linking. Projected square patterns show hydrogel microlenses of FIG. 13, row (a) that were incubated with 6.7 μM anti-biotin; row (b) were incubated with two (2) μM anti-biotin; row (c) were incubated with 1 μM anti-biotin; and row (d) were incubated with 0.6 μM anti-biotin. Thereafter, the hydrogel microlenses were exposed to differing levels of biocytin. For example, the hydrogel microlenses of the first column were exposed to PBS only (0 μM biocytin), the second column exposed to 0.1 μM biocytin, the third column exposed to 100 μM biocytin, and the fourth column exposed to one (1) mM biocytin. As expected, a higher concentration of biocytin is required to turn “on” the hydrogel microlenses initially incubated with higher concentrations of anti-biotin.

To observe microlens switching in more detail, we exposed the hydrogel microlenses to a narrower range of biocytin concentrations (FIGS. 13f-h). These experiments reveal that hydrogel microlenses show at least three distinct image projection modes, which we refer to here as “off,” “intermediate,” and “on,” in response to different anti-biotin concentrations (FIGS. 13f, 13g, and 13h, respectively). FIG. 13e shows the occurrence of each state upon changing the biocytin concentration as a function of the initial concentration of anti-biotin. In this manner, the bioresponsive microlenses display a transition range of finite width and are hence not purely binary response elements, thereby making the ultimate sensitivity of the element coupled to the ability to observe subtle changes in microlens focal length. Importantly, FIG. 13e shows modulation of microlens sensitivity over about four orders of magnitude, illustrating the potential for using gel swelling thermodynamics to modulate the sensitivity of a bioaffinity based sensor element.

In addition, FIG. 13d shows a lens incubated with 0.6 μM anti-biotin, which is insufficient to turn the lens “on”, even in PBS that is lacking added biocytin. These results suggest that the digital nature of an individual microlens response can be overcome by creating microlens arrays where individual array elements possess differing analyte sensitivities. These results are important to show that assays and structures of the instant invention are quantitative as well as qualitative. Utilizing the method of the instant invention, assays and structures of the instant invention can be utilized in, for example, but not limited to, high-throughput screening applications, drug testing applications, or genetic disease screening. The technology of the instant invention can be incorporated into a hand-held device for performing these and other applications and screens.

Biotin-functionalized hydrogel microlenses can be used to assay avidin and polyclonal anti-biotin using a brightfield optical microscopy technique. The hydrogel microlens assay can be easily constructed in inexpensive, simple, and rapid fashion, with high selectivity. The unique characteristics of the assay technology include the ability to determine the presence of an expected protein by monitoring the focal length of the microlens without the need for covalent tagging of the protein of interest. Furthermore, these microlenses can individually represent pixels in a biochip-type format, where such a chip can be read-out by simple optical microscopy coupled with image recognition software, again in a label-free format. Furthermore, we have demonstrated a new paradigm in label-free biosensing by combining antibody:antigen cross-linked hydrogel microlenses with a simple brightfield optical microscopy technique. The utility of the construct for small molecule detection, its resistance to interferences from non-specific adsorption, the ability to tune the sensitivity, small sample volume requirement, and the inexpensive, rapid and simple fabrication method make this a potentially powerful and generalizable biosensing construct. Furthermore, these fundamental advantages make this material attractive for the future development of bioresponsive materials in applications far beyond bioanalysis.

These fundamental advantages make this new technique attractive for the future development of a displacement type biointeractive pair assay where “on” and “off” signals are sufficient for primary affinity screening.

### Materials for Specific Embodiments

All reagents were purchased from Sigma-Aldrich (St. Louis, Mo., USA) unless otherwise specified. N-Isopropylacrylamide (NIPAm) was re-crystallized using hexanes (J. T. Baker) prior to use. N,N′-Methylene(bisacrylamide) (BIS) and ammonium persulfate (APS) were used without further purification. Acrylic acid (AAc) was distilled under reduced pressure. The glass cover slips, used as substrates were 24×50 mm Fisher Finest brand cover glass obtained from Fisher Scientific. 3-Aminopropyltrimethoxysilane (APTMS) was used for the functionalization of the glass substrates. Absolute (200 proof) and 95% ethanol were used for various purposes in this investigation. 1-ethyl-3-(3-dimethylaminopropyl) carbodiimide (EDC) and biotin hydrazide were purchased from Pierce. Dimethyl sulfoxide (DMSO) was obtained from J. T. Baker. Polyclonal anti-biotin (raised in goat) were purchased from Sigma-Aldrich. Water was distilled and then deionized (DI) to a resistance of at least 18 MΩ (Barnstead Thermolyne E-Pure system) and then filtered through a 0.2 μm filter to remove particulate matter before use. For printing pattern, 3M transparency film and a Hewlett Packard LaserJet 4000N printer was used.

Free-radical precipitation polymerization was used to synthesize microgels with a total monomer concentration of 300 mM, having molar composition of 89.4% NIPAm, 0.5% BIS, 10% AAc, 0.1% 4-acrylamido-fluorescein. In a typical procedure, 100 mL of filtered aqueous solution of NIPAm and BIS was added and the mixture was heated to approximately 70° C. under a N₂ atmosphere while stirring with a magnetic stir bar in a three-neck, 200 mL round-bottom flask. After one (1) hour, AAc and 4-acrylamido-fluorescein were added to the flask and polymerization was immediately initiated by injecting one (1) ml of a hot (approximately 70° C.) APS solution (6.13 mM). The reaction was continued at 70° C. for 4 hours under a N₂ environment. Particle purification was performed by dialysis against water for approximately two (2) weeks with the water being changed twice per day, using 10000 MW cut-off dialysis tubing (VWR).

Carbodiimides such as 1-ethyl-3-(3-dimethylaminopropyl) carbodiimide (EDC) and dicyclohexylcarbodiimide (DCC) were used to functionalize the anionic microgel with biotin and 4-aminobenzophenone (ABP), respectively. The functionalization was done by consuming 50% of the carboxyl groups on the particles by biotin and other 50% by ABP. Some portion of AAc groups are expected to remain available for binding to the cationic glass substrate because the reaction efficiency of the carbodiimide coupling is less than 100%. Biotinylation of 10-fold 2-[N-morpholino]ehtanesulfonic acid (MES) (pH 4.7) diluted anionic microgel (one (1) mL) was done by adding biotin hydrazide (3.8 mg dissolved in 0.5 mL of dimethyl sulfoxide (DMSO), 50% of the total amount of acrylic acid in the microgel solution) to the dilute microgel solution. To activate the coupling reaction EDC (15 mg) was added to the microgel and biotin solution. The reaction was carried out while stirring overnight at 4° C. Unreacted biotin hydrazide was removed by several cycles of centrifugation followed by resuspension in PBS (pH 7.5). The biotinylated acrylic acid microgel particles were then modified by ABP using DCC as the coupling reagent. Biotinylated acrylic acid microgel particle solution (one (1) mL in PBS (pH 7.5)) was centrifuged and redispersed in DMSO several times to replace the solvent from buffer medium to DMSO and finally redispersed in 700 μL of DMSO. Further, 150 μL of 0.01 M ABP in DMSO was added to the microgel solution followed by the addition of 150 μL of 0.01 M DCC in DMSO, making the total volume of reaction solution to 1 mL. The reaction was carried out overnight at room temperature while stirring in dark. Adding 0.5 mL de-ionized water to the reaction solution and filtration of the precipitated solid resulted in removal of insoluble side products from the microgel solution. The filtrate was subjected to several centrifugation cycles (centrifugation and resuspension) to remove any soluble reactant or side products. Finally, the solvent was replaced by PBS buffer (pH 7.5) by several more centrifugation cycles.

The following protocol was used to prepare the bioresponsive hydrogel microlens substrate. First, glass cover slips were treated in an Ar plasma (Harrick Scientific) for 30 minutes to remove any organic residuals from the glass surface. Plasma treatment was followed by immersion of the glass substrates in an ethanolic (absolute ethanol) 1% APTMS solution for approximately two (2) hours, after which they were removed from the solution and rinsed several times with 95% ethanol. These silane functionalized glass substrates were stored in 95% ethanol for no longer than five (5) days prior to use. Prior to assembly the substrates were rinsed with DI water and dried by a stream of N₂ gas. The silane functionalized glass substrate was then exposed to an aqueous 10% (v/v dilution of initial concentration following synthesis) biotin-ABP functionalized microgel solution buffered by 10 mM PBS buffer (pH 7.5). After 30 minutes, the substrate was rinsed with DI water, and dried with N₂ gas to leave behind microgels that are strongly attached due to Coulombic interactions to the substrate. A microlens array/silicone gasket/coverslip sandwich assembly was prepared and into the void space, buffered solution of polyclonal anti-biotin was introduced. The substrate was rinsed and the medium was replaced with PBS buffer (pH 7.5) after three (3) hours of incubation. The antigen-bound antibody was photo-ligated using the microgel-tethered ABP via UV irradiation using a 100 W longwave UV lamp for 30 minutes while cooling the coverslip on an ice bath. Various biocytin (60 μL) buffered in 10 mM PBS were introduced into the void space of the assembly for microscopic investigations as the microlenses response to competitive protein binding in time-dependant fashion.

Brightfield and fluorescence optical microscopies were used to monitor the bioresponsivity of hydrogel microlenses. Brightfield transmission and differential interference contrast (DIC) optical microscopies were used to study the changes in the optical properties of the hydrogel microlens attached to the substrate. An Olympus IX70 inverted microscope equipped with a high numerical aperture, oil immersion 100× objective (NA=1.30) was used for all microscopies reported here. Images were captured using either a black/white CCD camera (PixelFly, Cooke Corporation).

The examples herein are presented as illustrative and are not intended to limit the spirit or scope of the invention. The scope of the invention is set forth in the following claims.

1-43. (canceled)

44. A structure for detecting a target, comprising:
a functionalized polymer matrix having a boundary surface, wherein the boundary surface has chemically reactive groups; and
at least one biomolecule attached to at least one of the chemically reactive groups to form a surface-modified bioresponsive polymer matrix,
wherein the target is detected by a binding event between the target and the surface modified bioresponsive polymer matrix

45. The structure of claim 44, wherein the functionalized polymer matrix comprises natural and synthetic polymers.

46. The structure of claim 44, wherein the surface-modified bioresponsive polymer matrix comprises a microlens.

47. The structure of claim 46, wherein the microlens further comprises a hydrogel.

48. The structure of claim 44, wherein the at least one biomolecule comprises a protein, a polypeptide, or a nucleic acid molecule.

49. The structure of claim 44, wherein the structure can detect a target comprising a sugar, a protein, a nucleic acid, a hormone, a vitamin, a co-factor, a drug, an antibody, an antigen, an enzyme, or an ion.

50. The structure of claim 44, wherein the at least one biomolecule comprises at least two biomolecules that forms a biointeractive complex, wherein the biointeractive complex comprises protein:protein, protein:ligand, oligonucleotide:oligonucleotide, oligonucleotide: protein, oligonucleotide:ligand, antibody:antigen, enzyme:substrate, or protein:drug complexes.

51. The structure of claim 44, wherein detection of the binding event between the target and the surface-modified bioresponsive polymer matrix induces a detectable conformational change in the polymer matrix.

52. A method of detecting a target, comprising:
polymerizing monomers to form a polymer matrix having a boundary surface;
functionalizing the boundary surface of the polymer matrix using a functionalizing agent to produce chemically active groups;
attaching one or more biomolecules to the functionalized matrix to form a surface-modified bioresponsive polymer matrix ; and
detecting the target by a noncovalent binding event between the target and the surface-modified bioresponsive polymer matrix.

53. The method of claim 52, further comprising placing the polymer matrix onto a substrate to form a microlens

54. The method of claim 53, further comprising exposing the polymer matrix to a liquid to form a hydrogel.

55. The method of claim 52, wherein the one or more biomolecules comprise a protein, polypeptide, or nucleic acid molecule.

56. The method of claim 52, wherein the one or more biomolecules comprise a non-covalently associated biointeractive complex, wherein the biointeractive complex comprise protein:protein, protein:ligand, oligonucleotide:oligonucleotide, oligonucleotide:protein, oligonucleotide:ligand, antibody:antigen, enzyme: substrate, or protein:drug complex.

57. The method of claim 52, further comprising detecting the target by disrupting the biointeractive complex and inducing a detectable conformational change in the polymer matrix.

58. An assay system for detecting and measuring the concentration an analyte, comprising:
a functionalized polymer matrix having a boundary surface, wherein the boundary surface has chemically reactive groups;
at least one biomolecule attached to the chemically reactive groups to form a surface-modified bioresponsive polymer matrix;
a container for holding the surface-modified bioresponsive polymer matrix; and
a detection means for detecting a change in the bioresponsive polymer matrix in response to the analyte.

59. The assay system of claim 58, wherein the functionalized polymer matrix comprises a dried powder, a swollen gel, or a hydrogel.

60. The assay system of claim 58, wherein the at least one biomolecule comprises at least one non-covalently-interacting biointeractive complex, wherein the biointeractive complex comprises protein:protein, protein:ligand, oligonucleotide:oligonucleotide, oligonucleotide: protein, oligonucleotide:ligand, antibody:antigen, enzyme:substrate, or protein:drug complex.

61. The assay system of claim 58, wherein the container comprises a pipette tip, a micropipette tip, a plate well, or a centrifuge tube.

62. The assay system of claim 58, wherein the analyte comprises a sugar, a protein, a nucleic acid, a hormone, a vitamin, a co-factor, a drug, an antibody, an enzyme, or an ion.

63. The assay system of claim 58, wherein the detection means detects analyte disruption of the biointeractive complexes via controlled disassembly of colloidal gels, visualization of microgel-based microlenses, or particle counting following substrate deposition.
